(19) United States
(12) Patent Application Publication
(10) Pub. No.: US 2008/0146499 A1
(43) Pub. Date: Jun. 19, 2008

(54) Identification of the Precise Amino Acid Sequence of the Epitope Recognized by the Potent Neutralizing Human Anti-Hiv-1 Monoclonal Antibody Igg1b12

(76) Inventors: Jillian L.M. Waruk, Manitoba (CA); Jody D. Berry, Manitoba (CA); Blake T. Ball, Manitoba (CA); Francis A. Plummer, Manitoba (CA)

Correspondence Address: ADE & COMPANY INC., 2157 Henderson Highway, WINNIPEG, MB, R2G1P9, omitted

(21) Appl. No.: 10/599,734

(22) PCT Filed: Apr. 11, 2005
(86) PCT No.: PCT/CA05/00547

§ 371 Date: Nov. 28, 2007

(51) Int. Cl. A61K 38/08; C07K 14/005; A61K 38/10; C07K 16/00; A61P 31/12; C12Q 1/70; A61K 38/16; C07K 7/00

(52) U.S. Cl. 514/12; 530/329; 530/328; 530/327; 530/326; 530/325; 530/324; 514/17; 514/16; 514/15; 514/14; 514/13; 530/387.9; 435/5

(57) ABSTRACT

The IgG1b12 human monoclonal antibody binds gp120 strongly, and is the most potent HIV neutralizing antibody known. The exact amino acid sequence of this binding site is not known. We identified the minimal terminal peptide parts of the HIV-1 gp120 (envelope) protein, which IgG1b12 binds to. We did this by quadrupole time of flight (QqTOF) mass spectrometry using epitope excision. Epitope excision allows for determination of conformational epitopes. To do this we bound gp120 to IgG1b12, digested off all unbound portions of gp120, and then ran mass spectrometry of the resulting IgG1b12-gp120 peptide complex. The results allow us to determine the exact amino acid sequence involved in IgG1b12-gp120 interaction.

### FIELD OF THE INVENTION

The present invention relates generally to the field of medical treatments.

### BACKGROUND OF THE INVENTION

Global eradication of HIV will likely not occur with some miracle drug, but with a vaccine. The development of a safe, effective HIV vaccine has eluded scientists for over a decade. As the standard approaches to vaccine design are being exhausted, it is evident that in order to combat such a complex virus, newer technologies may hold the key to a successful vaccine. Until very recently, biology and mass spectrometry have been two facets of science without overlap. In the last few years, it has become apparent that the tools of mass spectrometry may be exploited to determine the specificities of the immune system receptors—from antibodies to toll like receptors—in order to gain information on the human immune response (3,4). This information can be applied to vaccine and therapeutics design, reagent development and so on.

In order to understand how to combat the HIV virus, we must study cases where individuals have combated HIV relatively successfully. In 1991, Burton, et al (5) cloned a clade B HIV-1 specific Fab fragments from an antibody library from the B cells of an HIV positive American male who had remained asymptomatic for over 6 years. They were then able to show that a certain monoclonal Fab antibody, b12, was a potently neutralizing antibody (6). Several years later, the crystal structure of the whole IgG1b12 molecule was resolved. It was determined that the docking site of IgG1b12 blocks the CD4 binding site of the HIV-1 surface glycoprotein termed gp120 (7). Gp120 amino acids involved in gp120-CD4 binding, as determined by x-ray crystallography, include Asp 368 (FIG. 3. D362), Glu 370 (G364) and Trp 427 (W484) (8). Gp120 amino acids determined to be involved in gp120-IgG1b12 binding are, according to the sequence in FIG. 3, G371, D373, P374, I376 and Y389.

The neutralizing capabilities of IgG1b12 have been described by many groups for many strains of HIV-1, and under many conditions (refs. 6,9-24). In vitro and ex vivo neutralization assays have been performed on primary isolates and lab-adapted strains from clades A, B, C, D, E, F and O (refs. 11, 16-24). Mouse (refs. 12, 14) and macaque (15) studies have shown that IgG1b12 can protect these animals from HIV-1 and SHIV challenges, respectively. In September of 2002, Lewis et al showed that neutralizing antibody could be found in the serum of mice receiving the IgG1b12 antibody gene delivered to the muscle by a recombinant adeno-associated virus (13). It has been shown that IgG1b12 can block HIV-1 attachment to CD4+ cells (10), as well as dendritic cell infection and transfer to T cells (9). There is no doubt that IgG1b12 is an invaluable tool for vaccine research and development.

The abilities of a vaccine to elicit immune responses that block viral infection of target cells and/or replication within these cells are critical to its success. Antibodies are capable of combating invading virus in many ways. When HIV-1 exits an infected cell, it acquires its envelope from that cell's membrane. Gp120 is therefore expressed on the surface of infected cells containing replicating virus. Gp120 may also exist on the surface as a result of HIV fusion to the cell membrane. Antibody can bind to the gp120 and mediate antibody dependant cellular cytotoxicity (ADCC), or compliment-dependant cytotoxicity (CDC) of the infected cell, or potentially block viral release. Perhaps the most important and exciting function that a protective antibody may have is its capacity to yield sterilizing immunity. Antibodies can bind to surface proteins on the virus and specifically block virus particles required for cell invasion. Higher affinity antibodies will remain tightly attached to the viral surface, out-competing for binding by the target cell receptor molecule(s) and are in general more powerfully neutralizing. Antibody-bound viruses can also trigger complement-mediated virolysis or phagocytosis (25). Virus that has entered the body is unable to infect target cells because neutralizing antibody mops up free virus which is then cleared by normal mechanisms. Infection cannot be established, and the host remains healthy. In contrast, cytotoxic T cells can only be specifically activated after an infection has been established and cells begin presenting antigen. Sterilizing immunity does not result in this case, and it is important to remember that once HIV has infected cells, it can begin to mutate and evolve to escape the immune response. Antibody-focused vaccine researchers strive to create a vaccine that will elicit a sterilizing antibody response. It is also important to induce cross clade-specific antibody responses so that the vaccine recipient is protected from infection by HIV of any clade.

One of the most important HIV-related phenomena to have been discovered in the last ten years is the existence of individuals who are resistant to HIV infection. This model of protection may hold the secret to the specific and/or innate immune responses required to successfully block HIV infection. Our group has identified a group of Kenyan female sex workers who, despite repeated exposure to HIV, remain uninfected (26). HIV-1 gp120-specific IgA has been isolated from the cervix of these women. The cervical IgA not only neutralizes HIV, but it can also inhibit the transcytosis of HIV across human epithelial cells (28). These women are exposed to HIV through heterosexual contact, therefore HIV initially comes into contact with cells of the genital tract. The virus must pass through epithelial cells via transcytosis in order to establish an infection. It is therefore plausible that neutralizing, transcytosis-inhibiting antibody may play a crucial role in HIV resistance in these women. Any vaccine that could educe such antibodies may provide sterilizing immunity to its recipients.

The IgG1b12 antibody was cloned from an HIV+ donor who had been HIV+ for over 6 years. Why had he remained AIDS-free for over 6 years? It is very possible that his immune response was effective at combating the disease, keeping the virus ‘at bay’ for an extended period of time. HIV researchers continue to hash out the mechanism by which some individuals become long-term non-progressors. It is possible that the existence of potently neutralizing antibodies aids in harnessing the infection. The IgG1b12 epitope specificity may provide information, in the form of a marker, about those individuals who will not progress quickly to AIDS. Knowledge of what comprises a neutralizing epitope for antibodies may be applicable to clinical settings as well. For instance, if antibodies from patient ‘X’ recognized a specific sequence in the HIV envelope protein, they have an increased chance of being a long-term non-progressor. Doctors could use this information to tailor drug regimens specifically for each patient.

### SUMMARY OF THE INVENTION

According to a first aspect of the invention, there is provided a purified polypeptide, the amino acid sequence of which comprises at least 6 contiguous residues of any one of SEQ ID No. 1-6.

According to a second aspect of the invention, there is provided a method of immunizing an individual against HIV infection comprising administering to an individual a purified polypeptide, the amino acid sequence of which comprises at least 6 contiguous residues of any one of SEQ ID No. 1-6.

According to a third aspect of the invention, there is provided the use of a purified polypeptide as a vaccine, the amino acid sequence of which comprises at least 6 contiguous residues of any one of SEQ ID No. 1-6.

According to a fourth aspect of the invention, there is provided the use of a purified polypeptide as a medicament, the amino acid sequence of which comprises at least 6 contiguous residues of any one of SEQ ID No. 1-6.

According to a fifth aspect of the invention, there is provided a method of preparing an immune globulin effective against Human Immunodeficiency virus comprising:

vaccinating a plurality of donors with a purified polypeptide, the amino acid sequence of which comprises at least 6 contiguous residues of any one of SEQ ID No. 1-6;

isolating plasma from each of said donors after a period of time sufficient to allow production of antibodies against said polypeptide;

pooling the plasma; and

preparing an immune globulin from the pooled plasma.

According to a sixth aspect of the invention, there is provided a method of determining a course of treatment for an individual infected with human immunodeficiency virus comprising:

screening a sample from an individual infected with human immunodeficiency virus for antibodies binding to a purified polypeptide, the amino acid sequence of which comprises at least 6 contiguous residues of any one of SEQ ID No. 1-6,

wherein presence of antibodies against said polypeptide indicates that a less aggressive treatment is needed.

According to a seventh aspect of the invention, there is provided a method of treating an individual infected or suspected of being infected by human immunodeficiency virus comprising administering to said individual a therapeutically effective amount of a purified polypeptide, the amino acid sequence of which comprises at least 6 contiguous residues of any one of SEQ ID No. 1-6.

According to an eighth aspect of the invention, there is provided a method of treating an individual infected or suspected of being infected by human immunodeficiency virus comprising administering to said individual a purified polypeptide, the amino acid sequence of which comprises at least 6 contiguous residues of any one of SEQ ID No. 1-6.

### BRIEF DESCRIPTION OF THE DRAWINGS

FIG. 1. MALDI QqTOF mass spectrometry results of HIV-1 gp120 epitope mapping. IIIB (a) or MN (b) gp120 was bound to IgG1b12 antibody and was digested overnight with Glu-C endopeptidase. Protein fragments not bound by IgG1b12 were washed away. Antibody-bound fragments protected from digestion were analyzed by mass spectrometry. The size of the first peak, 1807 (mass/charge, or m/z), corresponds to the N-terminal sequence ATTTLFCASDAKAYDTE (as determined by the theoretical digest program). 1867, the second peak, corresponds to the sequence KLWVTVYYGVPVWK. The third peak, 2097 corresponds to the sequence TEKLWVTVYYGVPVWKE.

FIG. 2. MALDI QqTOF mass spectrometry results of gp120 epitope mapping. IIIB (a) or MN (b) gp120 was bound to IgG1b12 antibody and was digested overnight with trypsin endopeptidase. Protein fragments not bound by IgG1b12 were washed away. Antibody-bound fragments protected from digestion were analyzed by mass spectrometry. The size of the peak 1357 corresponds to the N-terminal sequence EATTTLFCASDAK. The peaks at 1609 corresponds to the N-terminal sequence LWVTVYYGVPVWK.

FIG. 3. Amino acid sequence of HIV-1 gp120 IIIB (Immunodiagnostics, Inc.). Amino acids identified by mass spectrometry are underlined.

FIG. 4. Amino acid sequence of HIV-1 gp120 MN (Immunodiagnostics, Inc.). Amino acids identified by mass spectrometry are underlined. Bold G (glycine) is the unknown putative amino acid change G-T (threonine) recognized by the exquisite sensitivity of mass spectrometry (2097 peak).

FIG. 5. MALDI QqTOF mass spectrometry results of HIV-1 gp120 epitope mapping. Amino terminus and CD4 binding site gp120 peptides were bound to IgG1b12 antibody.

FIG. 6. Mass spectrometry results of 2 hour N-term peptide digestion with trypsin. The peak at m/z=1737 corresponds to the amino acids that remained bound to the IgG1b12 after trypsin digest. The 1737 peak-corresponds to the digested fragment amino acid sequence KLWVTVYYGVPVWK.

FIG. 7. Consensus sequence for IgG1b12 binding.

FIG. 8. Epitope mapping confirmation that the IgG1 b12 epitope on glu-C digested gp120 is variable region-specific. Gp120 MN was incubated with either IgG1 b12 (A) or KZ52 control (B) antibodies linked to Sepharose beads. The antigen-antibody complex was digested with the endoprotease glu-C, washed and tested by MALDI QqTOF mass spectrometry for bound epitopes.

FIG. 9. Epitope excision mapping by trypsin digestion of gp120 confirms that the IgG1 b12 epitope recognition by gp120 is variable region-specific. Gp120 MN was incubated with either (a) IgG1 b12 or (b) KZ52 control antibodies linked to Sepharose beads. Unbound antigen was washed away and bound gp120 was digested with the endoprotease trypsin, washed and tested for antigen-antibody interactions.

FIG. 10. Western blot of IgG1 b12 shows that IgG1 b12 binds whole, denatured gp120. Soluble gp120 was resolved on SDS-PAGE gel and b12 binding was detected by chemiluminescence. The first lane shows b12 binding to soluble gp120, lane 2 shows reactivity of the secondary antibody alone vs. gp120 (negative control), lane 3 shows b12 binding to BSA (negative control).

FIG. 11. IgG1b12 recognizes synthetic peptide sequence from the amino-terminus of gp120, and not a scrambled version of the same peptide. ELISA plates were coated with IgG1 b12, biotinylated N-term and scrambled peptides were added and tested for binding. Representative data from one of three experiments is shown.

FIG. 12. The binding of N terminal peptide to IgG1 b12 can be blocked by soluble gp120. ELISA plates were coated with IgG1 b12. Soluble gp120 was added then biotinylated peptide was added and tested for binding. Representative data from one of 3 experiments is shown.

FIG. 13. Antigen recognized by differentially immunized mice. Four groups of 4 mice were immunized on five separate occasions with adjuvant plus PBS alone, gp120, N terminal peptide, or scrambled peptide. Serum was collected and tested for antibody recognition of gp120 (A), N terminal peptide (B), and scrambled peptide (C) by indirect ELISA. Representative data from one of 3 experiments is shown.

### DESCRIPTION OF THE PREFERRED EMBODIMENTS

Unless defined otherwise, all technical and scientific terms used herein have the same meaning as commonly understood by one of ordinary skill in the art, to which the invention belongs. Although any methods and materials similar or equivalent to those described herein can be used in the practice or testing of the present, invention, the preferred methods and materials are now described. All publications mentioned hereunder are incorporated herein by reference.

### Definitions

As used herein, “effective amount” refers to the administration of an amount of a given compound that achieves the desired effect.

As used herein, “purified” does not require absolute purity but is instead intended as a relative definition. For example, purification of starting material or natural material to at least one order of magnitude, preferably two or three orders of magnitude is expressly contemplated as falling within the definition of “purified”.

As used herein, the term “isolated” requires that the material be removed from its original environment.

As used herein, the term “treating” in its various grammatical forms refers to preventing, curing, reversing, attenuating, alleviating, minimizing, suppressing or halting the deleterious effects of a disease state, disease progression, disease causitive agent other abnormal condition.

As described herein, the region recognized by IgG1b12, which is the most potent antibody yet described which is capable of neutralizing HIV-1, has been identified. Furthermore, the neutralizing ability of IgG1b12 is likely involved in protective immune responses to HIV-1 and this can be induced in others to generate protective HIV-1 specific responses. For example, the sequence of this particular epitope could be used in blocking HIV infection. Knowledge of when/how responses to this epitope develop may also be useful in tailoring alternate therapeutic interventions, as discussed below.

As discussed below, the minimal epitopes as defined by Glu-C digestion are:

LWVTVYYGVPVWKE
and
ATTTLFCASDAK

while the minimal epitopes as defined by Trypsin digestion are:

LWVTVYYGVPVWK
and
EATTTLFCASDAK

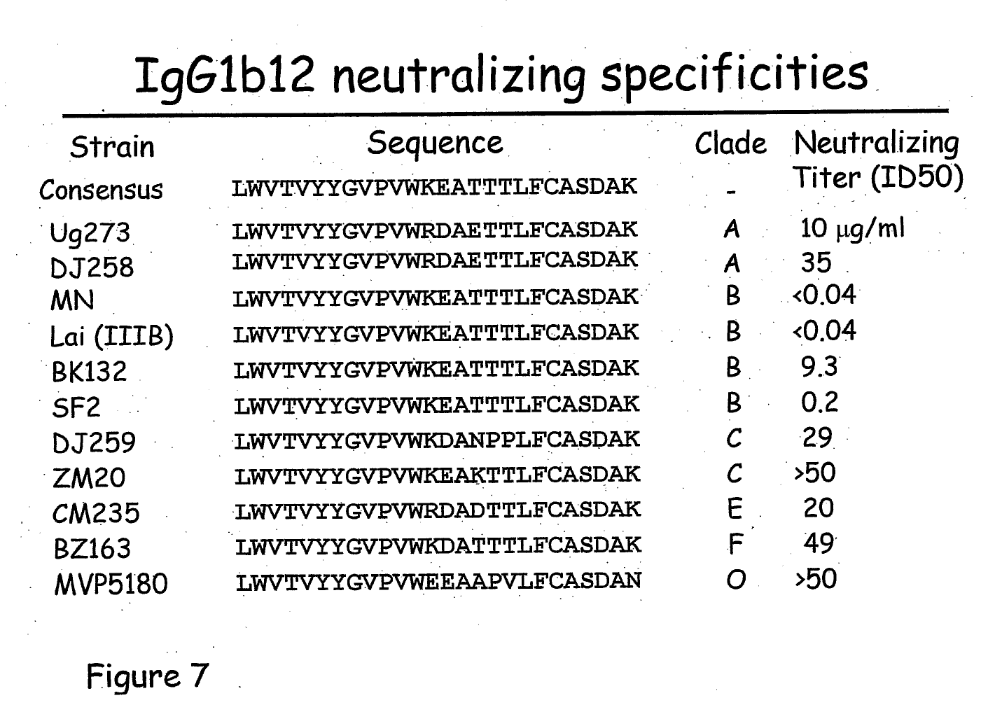

This leads to a consensus sequence for IgG1b12 binding of LWVTVYYGVPVWKEATTTLFCASDAK (SEQ ID No. 1, shown in FIG. 7) and a sequence of GVPVWKEATTTL (SEQ ID No. 2). As can be seen in FIG. 7, the sequence of this region varies somewhat in different strains and clades.

In one embodiment of the invention, there is provided an isolated and/or purified polypeptide, the amino acid sequence of the polypeptide comprised of or consisting essentially of 6 or more consecutive residues of SEQ ID No. 1 or SEQ ID No. 2, that is, LWVTVYYGVPVWKEATTTLFCASDAK or GVPVWKEATTTL or a variant thereof, for example, as shown in FIG. 7. In other embodiments, the polypeptide may consist of 7 or more consecutive residues, 8 or more consecutive residues, 9 or more consecutive residues or 10 or more consecutive residues of SEQ ID No. 1, or SEQ ID No. 2, that is, LWVTVYGVPVWKEATTTLFCASDAK or GVPVWKEATTTL or a variant thereof.

As will be apparent to one of skill in the art, as used herein, “variant thereof” refers to peptides derived from or based on the amino acid sequence from the same region of gp120 from a different clade or isolate of HIV that act as a neutralizing peptide, as discussed below. Examples of such variants are shown in FIG. 7. Other potential variants can readily be determined using means known in the art and any suitable database containing gp120 sequences.

Thus, in another embodiment of the invention, there is provided an isolated and/or purified polypeptide, the amino acid sequence of the polypeptide comprised of or consisting essentially of 6 or more consecutive residues of SEQ ID No. 3 or SEQ ID No. 4, that is, LWVTVYYGVPVW(E/K/R)(E/D)A(E/T/N/K/D/A)(T/P)(T/P/V)LFCASDAK or GVPVW(E/K/R)(E/D)A(E/T/N/K/D/A)(T/P)(T/P/V)L or a variant thereof, for example, as shown in FIG. 7. In other embodiments, the polypeptide may consist of 7 or more consecutive residues, 8 or more consecutive residues, 9 or more consecutive residues or 10 or more consecutive residues of SEQ ID No. 3 or SEQ ID No. 4, that is, LWVTVYYGVPVW(E/K/R)(E/D)A(E/T/N/K/D/A)(T/P)(T/P/V)LFCASDAK or GVPVW(E/K/R)(E/D)A(E/T/N/K/D/A)(T/P)(T/P/V)L or a variant thereof.

In yet another embodiment of the invention, there is provided an isolated and/or purified polypeptide, the amino acid sequence of the polypeptide comprised of or consisting essentially of 6 or more consecutive residues of SEQ ID No. 5 or SEQ ID No. 6, that is, LWVTVYYGVPVW(E/K/R)(E/D)A(E/T/N/D)(T/P)(T/P)LFCASDAK or GVPVW(E/K/R)(E/D)A(E/T/N/K/D/A)(T/P)(T/P)L or a variant thereof, for example, as shown in FIG. 7. In other embodiments, the polypeptide may consist of 7 or more consecutive residues, 8 or more consecutive residues, 9 or more consecutive residues or 10 or more consecutive residues of SEQ ID No. 5 or SEQ ID No. 6, that is, LWVTVYYGVPVW(E/K/R)(E/D)A(E/T/N/D)(T/P)(T/P)LFCASDAK or GVPVW(E/K/R)(E/D)A(E/T/N/D)(T/P)(T/P)L or a variant thereof.

In yet another embodiment, there is provided an isolated and/or purified polypeptide consisting of or consisting essentially of SEQ ID No. 1, SEQ ID No. 2, SEQ ID No. 3, SEQ ID No. 4, SEQ ID No. 5 or SEQ ID No. 6.

Furthermore, it is of note that it is well known in the art that some modifications and changes can be made in the structure of a polypeptide without substantially altering the biological function of that peptide, to obtain a biologically equivalent polypeptide. In one aspect of the invention, the above-described peptides may include peptides that differ by conservative amino acid substitutions. The peptides of the present invention also extend to biologically equivalent peptides that differ by conservative amino acid substitutions. As used herein, the term “conserved amino acid substitutions” refers to the substitution of one amino acid for another at a given location in the peptide, where the substitution can be made without substantial loss of the relevant function, in this case, the folding of the epitope. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. It is of note that one of skill in the art would anticipate that unconserved or not highly conserved amino acids are more likely candidates for substitution without loss of function.

In some embodiments, conserved amino acid substitutions may be made where an amino acid residue is substituted for another having a similar hydrophilicity value (e.g., within a value of plus or minus 2.0), where the following may be an amino acid having a hydropathic index of about −1.6 such as Tyr (−1.3) or Pro (−1.6)s are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (0); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In alternative embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: Ile (+4.5); Val (+4.2); Len (+3.8); Phe (+2.8); Cys (+2.5); Met (+1.9); Ala (+1.8); Gly (−0.4); Thr (−0.7); Set (−0.8); Trp (−0.9); Tyr (−1.3); Pro (−1.6); His (−3.2); Glu (−3.5); Gln (−3.5); Asp (−3.5); Am (−3.5); Lys (−3.9); and Arg (−4.5).

In alternative embodiments, conserved amino acid substitutions may be made where an amino acid residue is substituted for another in the same class, where the amino acids are divided into non-polar, acidic, basic and neutral classes, as-follows: non-polar: Ala, Val, Len, Ile, Phe, Trp, Pro, Met; acidic: Asp, Glu; basic: Lys, Arg, His; neutral: Gly, Ser, Thr, Cys, Asn, Gln, Tyr.

As will be apparent to one of skill in the art, it is also possible that the b12 antibodies are recognizing a conformational site within the above-described sequence, for example, a conformational epitope formed by non-adjacent, that is, non-contiguous residues. As such, in these embodiments, the peptide may comprise 2 or more contiguous amino acids from a first region of SEQ ID No. 1 separated by a linker of variable sequence to 2 or more contiguous amino acids from a second region of SEQ ID No. 2. As will be appreciated by one of skill in the art, the linker does not necessarily need to correspond verbatim to the intervening residues between the two regions but should be such that the confirmation of the residues within the conformational epitope is approximated. That is, in other embodiments, there is provided a peptide comprising 2 or more amino acids from the N-terminus region of any one of the peptides according to SEQ ID No. 1, 3 or 5 fused to 2 or more amino acids from the C-terminal region of any one of the peptides according to SEQ ID No. 1, 3 or 5 separated by a linker of a length that corresponds to the number of amino acids separating the N-terminal region amino acids from the C-terminal region amino acids in the native sequence.

In another embodiment of the invention, there is provided a method of immunizing an individual against HIV infection comprising administering to an individual an effective amount the isolated or purified polypeptide described above. As will be appreciated by one of skill in the art, the effective amount is an amount sufficient to induce an immune response within the individual. It is of note that when the purified polypeptide is used as a vaccine, the preparation may include at least one suitable excipient.

In these embodiments, administration of the polypeptide to an individual, for example, a human, results in the individual obtaining sterilizing immunity against the human immunodeficiency virus, as discussed below. Specifically, immunization will result in the production of neutralizing antibodies against the above-described polypeptide under subsequent challenge. That is, on subsequent exposure to gp120, antibodies will be produced which will bind to gp120 at the site required for binding to CD4, thereby preventing viral infection of T-cells and will also target the viral particles for removal by antibody dependant cellular cytotoxicity or complement dependent cytotoxicity.

In other embodiments, any one of the above-described peptides is administered to an individual infected with or suspected of being infected with Human Immunodeficiency virus. As discussed above, immunization will promote the production of neutralizing antibodies, which will in turn bind to gp120, thereby preventing viral infection of T-cells. Thus, immunization in these embodiments will slow disease progression by decreasing the rate of viral infection. As will be apparent to one of skill in the art, the immunization may be combined with other anti-HIV compounds, for example, azidothymidine (AZT), lamivudine (3TC), dideoxyinosine (ddi), dideoxycytidine (ddc) and ritonavir, as well as other reverse transcriptase and protease inhibitors.

In yet other embodiments, an immune globulin effective against Human Immunodeficiency virus may be prepared by vaccinating a plurality of donors with any one of the above-described isolated or purified polypeptides; isolating plasma from each of said donors after a period of time sufficient to allow production of antibodies against said polypeptide; pooling the plasma; and preparing an immune globulin from the pooled plasma using means known in the art. As will be appreciated by one of skill in the art, the immune globulin preparation may be used as a treatment for individuals having been recently infected or suspected of having been infected with human immunodeficiency virus. That is, antibodies within the immune globulin preparation will bind to gp120, preventing binding to CD4 and targeting the viral particles for removal as discussed above.

In another embodiment of the invention, there is provided a method of treating an individual infected or suspected of being infected by human immunodeficiency virus comprising administering to said individual a therapeutically effective amount of any one of the above-described purified polypeptides. In these embodiments, the above-described polypeptide interacts with CD4, effectively acting as a decoy substrate and preventing or greatly reducing gp120 binding to CD4 by occupying CD4 binding sites. As will be apparent to one of skill in the art, this treatment may be combined with other treatments known in the art as well as for example the immune globulin preparation described above.

In another embodiment of the invention, there is provided a method of determining a course of treatment for an individual infected with human immunodeficiency virus comprising screening a sample from an individual infected with human immunodeficiency virus for antibodies binding to any one of the above-described purified polypeptides, wherein presence of antibodies against said polypeptide indicates that a less aggressive treatment is needed. Specifically, as discussed herein, the presence of antibodies against this region of gp120 has been shown to result in non-progression of the disease. As a consequence, individuals having natural immunity against this specific region of gp120 may not need to be treated aggressively, as discussed below.

In some embodiments, any one of the above-described polypeptides may be combined with a suitable carrier peptide known in the art.

In some embodiments, the purified polypeptide or immune globulin may be combined with other compounds or compositions known in the art such that the is a pharmaceutical composition in the form of, for example, a pill, tablet, liquid, film or coating using means known in the art and as discussed below.

It is of note that the purified polypeptide or immune globulin discussed above may be prepared to be administered in a variety of ways, for example, topically, orally, intravenously, intramuscularly, subcutaneously, intraperitoneally, intranasally or by local or systemic intravascular infusion using means known in the art and as discussed below.

In some embodiments, the above-described pharmaceutical composition may be combined with a pharmaceutically or pharmacologically acceptable carrier, excipient or diluent, either biodegradable or non-biodegradable. Exemplary examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, mathacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecylsulfate, carboxymethylcellulose calcium, carboxymethylcellulose sodium, methylcellulose, .hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches. See, for example, Remington: The Science and Practice of Pharmacy, 1995, Gennaro ed.

As will be apparent to one knowledgeable in the art, specific carriers and carrier combinations known in the art may be selected based on their properties and release characteristics in view of the intended use. Specifically, the carrier may be pH-sensitive, thermo-sensitive, thermo-gelling, arranged for sustained release or a quick burst. In some embodiments, carriers of different classes may be used in combination for multiple effects, for example, a quick burst followed by sustained release.

In other embodiments, the above-described pharmaceutical composition at concentrations or dosages described above may be encapsulated for delivery. Specifically, the pharmaceutical composition may be encapsulated in biodegradable microspheres, microcapsules, microparticles, or nanospheres. The delivery vehicles may be composed of, for example, hyaluronic acid, polyethylene glycol, poly(lactic acid), gelatin, poly(E-caprolactone), or a poly(lactic-glycolic) acid polymer. Combinations may also be used, as, for example, gelatin nanospheres may be coated with a polymer of poly(lactic-glycolic) acid. As will be apparent to one knowledgeable in the art, these and other suitable delivery vehicles may be prepared according to protocols known in the art and utilized for delivery of the. Alternatively, the delivery vehicle may be suspended in saline and used as a nanospray for aerosol dispersion.

The above-described pharmaceutical compounds at therapeutically effective dosages would therefore reduce the spread of an HIV infection by accomplishing at least one of the following: decreasing viral load, preventing or limiting the rate of viral infection and preventing further infection by the virus.

The kits of the invention comprise one or more containers comprising a purified polypeptide or immune globulin as described above, a suitable excipient as described herein and a set of instructions, generally written instructions although electronic storage media (e.g., magnetic diskette or optical disk) containing instructions are also acceptable, relating to the use and dosage of the for the intended treatment. The instructions included with the kit generally include information as to dosage, dosing schedule, and route of administration for the intended treatment. The containers of the glandular kallikrein may be unit doses, bulk packages (e.g., multi-dose packages) or sub-unit doses.

The invention will now be described by way of examples; however, the invention is not limited by the examples.

Full length, purified IgG1b12 antibody was contributed by Carlos Barbas, III (The Scripps Research Institute, La Jolla, Calif., USA). The antibody was linked to cyanogen-bromide activated sepharose beads (Sigma-Aldrich, Oakville, Ontario). Epitope excision was performed to include potential conformational epitopes. The antibody-bead mixture was incubated with either MN or IIIB HIV-1 gp120 (ImmunoDiagnostics, Inc. Woburn, Mass.) or synthetic HIV-1 gp120 peptides (United Biochemical Research, Inc, Seattle, Wash., USA) for 2 hours under physiological conditions. After several washes, trypsin (Calbiochem-Novabiochem Corporation, San Diego, Calif., USA), or Glu-C (Roche Diagnostics Canada. Laval, Quebec) enzyme digests were performed. Unbound, digested material was washed away. Antibody-bound fragments protected from digestion were analyzed by mass spectrometry. Bead+antibody+peptide complex were spotted on a gold plate along with DHB matrix (Sigma-Aldrich, Oakville, Ontario) (3,4). The sample was analyzed on the prototypic QqTOF mass spectrometer by matrix-assisted laser desorption/ionization.

Theoretical MN and IIIB trypsin and Glu-C digests were performed on ProMac, which provided molecular masses-for each possible fragment created by enzyme digestion.

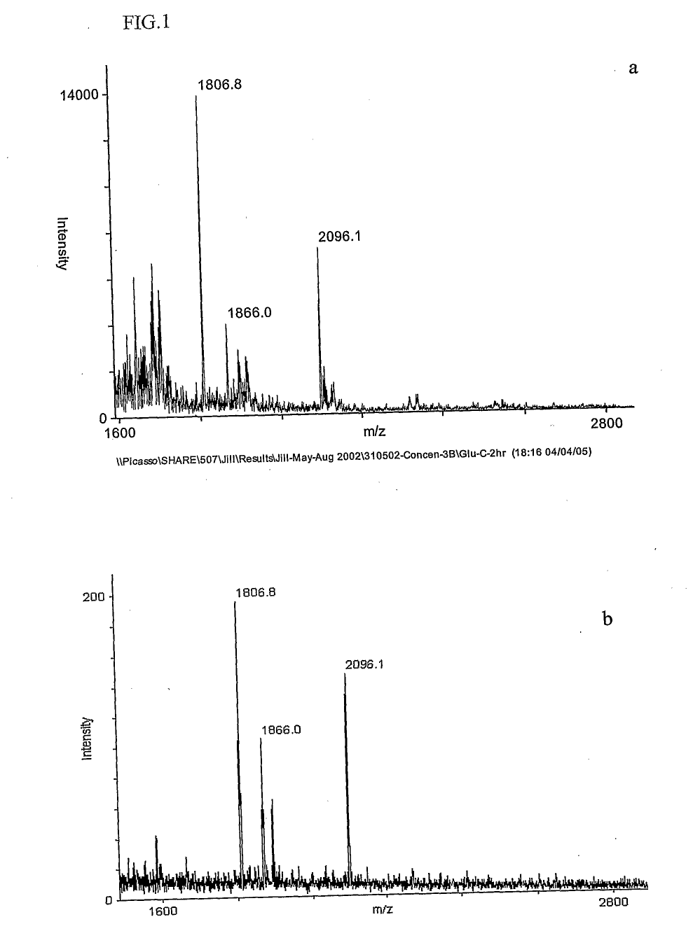

As shown in FIG. 1, IIIB (a) or MN (b) gp120 was bound to IgG1b12 antibody and was digested overnight with Glu-C endopeptidase. Protein fragments not bound by IgG1b12 were washed away. Antibody-bound fragments protected from digestion were analyzed by mass spectrometry. The size of the first peak, 1807 (mass/charge, or m/z), corresponds to the N-terminal sequence ATTTLFCASDAKAYDTE (as determined by the theoretical digest program). 1867, the second peak, corresponds to the sequence KLWVTVYYGVPVWK. The third peak, 2097 corresponds to the sequence TEKLWVTVYYGVPVWKE.

This is the first experiment describing a region other than the CD4-binding site as the IgG1b12 epitope. The CD4-binding site epitope expected peak size is 2067.01 m/z with the sequence QFGNNKTIIFKQSSGGDPE. This epitope is clearly missing, implying that it may not be involved in the specific interactions between IgG1b12 and gp120.

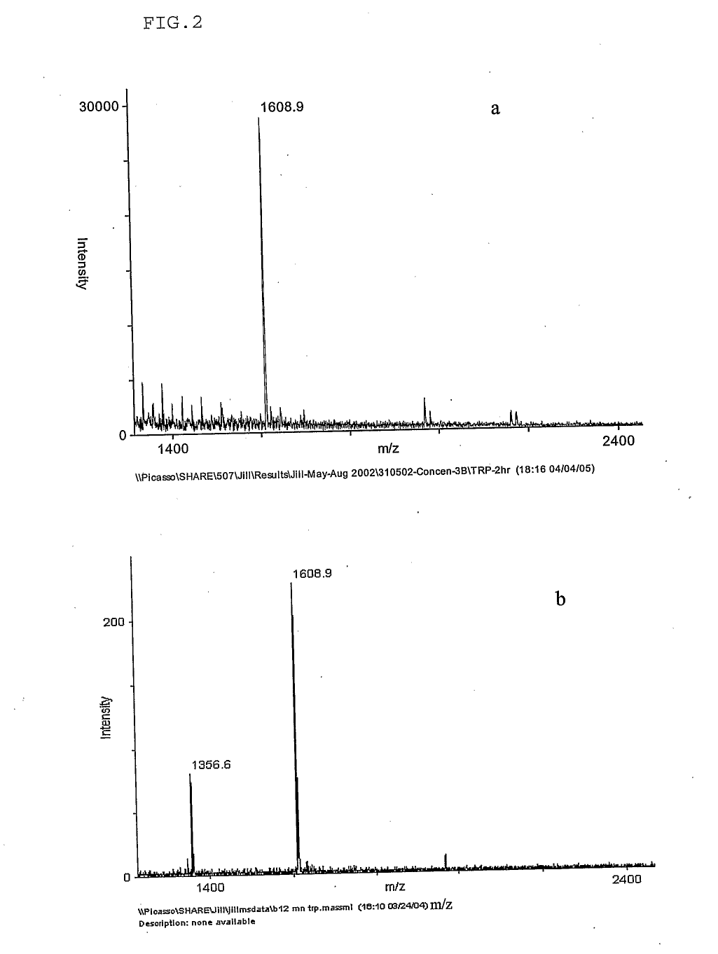

As can be seen in FIG. 2, IIIB (a) or MN (b) gp120 was bound to IgG1b12 antibody and was digested overnight with trypsin endopeptidase. Protein fragments not bound by IgG1b12 were washed away. Antibody-bound fragments protected from digestion were analyzed by mass spectrometry. The size of the peak 1357 corresponds to the N-terminal sequence EATTTLFCASDAK. The peak at 1609 corresponds to the N-terminal sequence LWVTVYYGVPVWK.

This indicates that the amino terminus is the source of the IgG1b12 epitope. The mass spectrometer has unequivocally identified the amino terminus as the epitope. The trypsin digest mass spectrometry results strengthens the results seen in the first figure.

FIG. 3 shows the amino acid sequence of HIV-1 gp120 IIIB (Immunodiagnostics, Inc.). Amino acids identified by mass spectrometry are underlined.

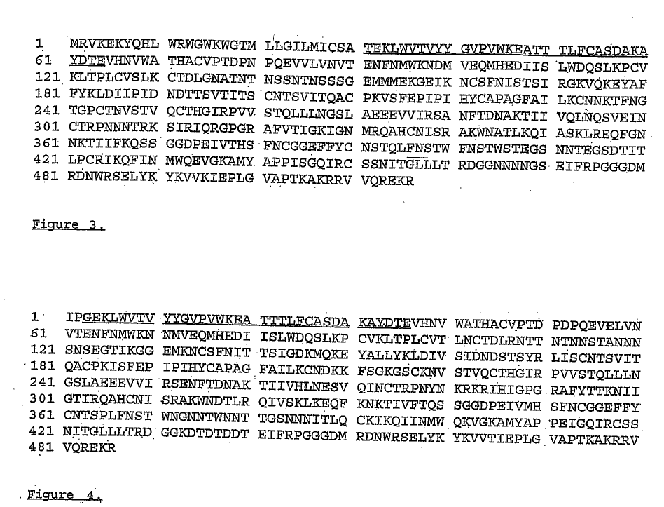

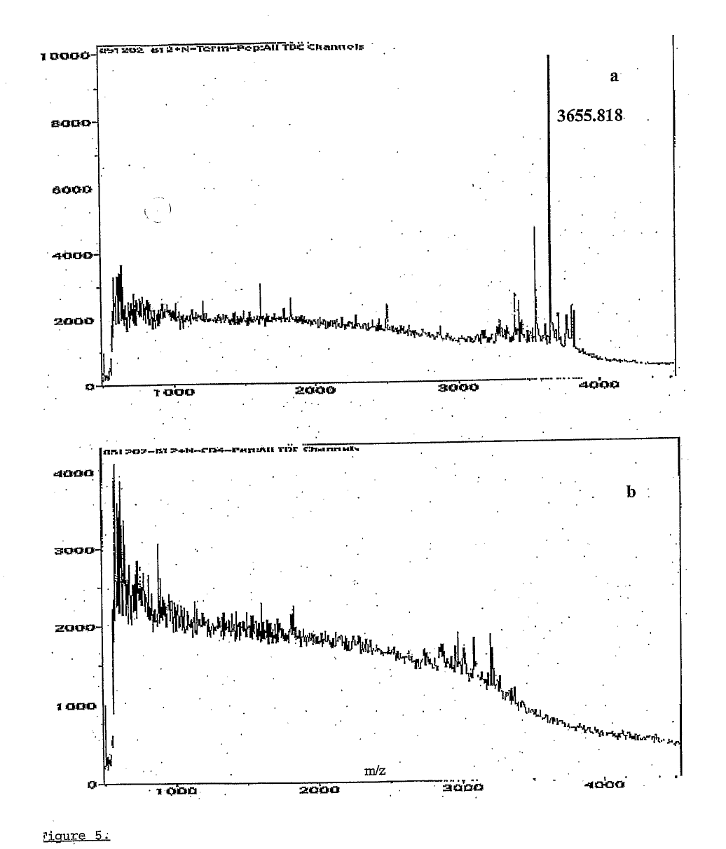

FIG. 4 shows the amino acid sequence of HIV-1 gp120 MN (Immunodiagnostics, Inc.). Amino acids identified by mass spectrometry are underlined. Bold G (glycine) is the unknown putative amino acid change G→T (threonine) recognized by the exquisite sensitivity of mass spectrometry (2097 peak).

It appears that IgG1b12 specifically interacts with amino acids within a 34 amino acid sequence near the amino terminus of HIV-1 gp120. It is unlikely that the antibody recognizes the entire linear sequence, because linear epitopes are normally half this length. The digestive cut between the 1867/1807 and 1609/1357 peaks suggests that this part of the peptide is not bound to the antibody, and therefore exposed to digestion. Interaction probably occurs between IgG1b12 and amino acids from both sequences.

Thus, as described above, mass spectrometry epitope mapping has identified the gp120 sequence involved in IgG1b12-binding. E. 0. Saphire et al (Science, 2001) initially used the computer program AutoDock to predict the gp120 amino acids involved in gp120-CD4 interactions. The software predicted that amino acids Ser³⁶⁵, Asp³⁶⁸, Ile³⁷¹, Tyr³⁸⁴, and Val⁴³⁰. According to our gp120 sequences IIIB and MN, these amino acids correspond to Ser³⁷⁰, Asp³⁷³, Ile³⁷⁶, and likely Val⁴³⁵ for the IIIB sequence, and Ser³⁴¹, Asp³⁴⁴, Ile³⁴⁷, and likely Val⁴⁰³ in the MN sequence. Saphire et al then confirmed their results by alanine mutation studies. These amino acids are near the CD4-binding site, and therefore it is assumed that IgG1b12 physically blocks the CD4 binding site. Although both of these methods of epitope mapping are commonly accepted and practiced, it is important to note that conclusions drawn from both these experiments about epitope sequence are purely based on inference. The computer program is entirely theoretical and the mutation studies only provide information on amino acids that play a role in binding, though this role may be conformational, and not direct contact. Our mass spectrometry epitope mapping experiments gave much different results than those of Saphire's group. Through mass spectrometry, we have identified the N-terminal gp120 sequence LWVTVYYGVPVWKEATTTLFCASDAK as the sequence containing the amino acids involved in IgG1b12 binding. According to the ImmunoDiagnostics IIIB gp120 sequence, these amino acids fall between Leu³⁴ and Lys⁵⁹, and the amino acids Leu⁶ and Lys³¹ in the MN sequence.

Because, as discussed above, previous experiments have suggested that residues near the CD4 binding site may be important for IgG1b12-gp120 interactions, further studies are required. It has been proposed that IgG1b12 may interact with an isoleucine residue that would otherwise be cleaved off our trypsin digested CD4 binding site peptide (. . . SSGGDPEI . . . ) (7). Epitope mapping experiments were performed using synthetic peptides. One peptide, the “N-term” (amino terminus) peptide was tested for it's binding to IgG1b12. The N-term sequence is as follows: KLWVTVYYGVPVWKEATTTLFCASDAKAYDTE. A second peptide covered the CD4 binding site, including the isoleucine (I) residue. The sequence QFGNNKTIIFKQSSGGDPEIVTHSFNCGGE was tested for antibody binding, and these results were measured by mass spectrometry, as shown in FIG. 5.

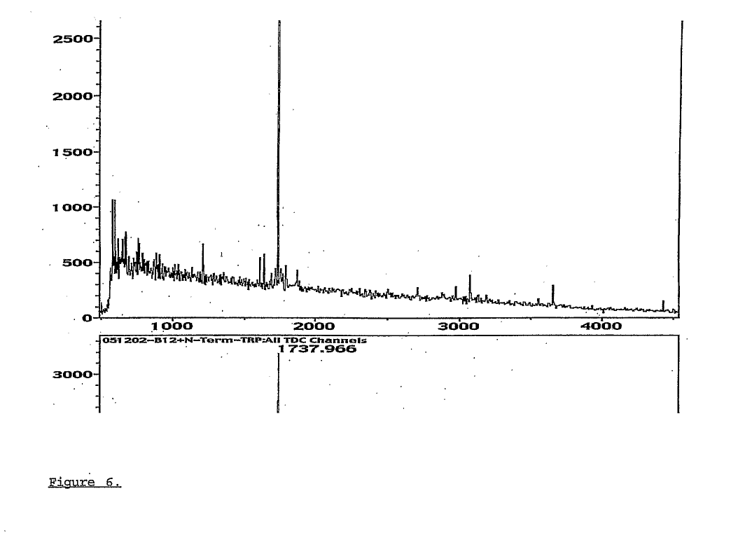

With the peptides as starting material, IgG1b12 still specifically recognizes the amino terminus and not the CD4 binding site. A 2-hour trypsin digest confirmed the identity of our peptide (FIG. 6). The 1737 peak corresponds to the digested fragment amino acid sequence KLWVTVYYGVPVWK.

Through mass spectrometry epitope mapping, using two different strains of HIV-1 envelope, two different endopeptidases, and synthetic peptides, it is clear that the IgG1b12-gp120 binding interface is not as was previously proposed. Though it is possible that the amino acids previously implicated in IgG1 b12 binding play an important role in bond formation, it is likely that this role is merely conformational. Mass spectrometry identification of epitopes is based strictly on identifying peptide regions that bind to antibody, and gives proof of those interactions. Most other antibody epitope mapping strategies rely on inference based on indirect observations. The epitope mapping experiments performed thoroughly identified the amino terminal sequence (TEKLWVTVYYGVPVWKEATTTLFCASDAKAYDTE) as the fragment that contains individual residues involved in IgG1b12-gp120 interactions.

Thus, as discussed above, the region recognized by IgG1b12, which is the most potent antibody yet described which is capable of neutralizing HIV-1 in multiple assays, has been identified. Furthermore, the neutralizing ability of IgG1b12 is likely involved in protective immune responses to HIV-1 and this can be induced in others to generate protective HIV-1 specific responses. Furthermore, knowledge of the sequence of this particular epitope could be used in blocking HIV infection. Knowledge of when/how responses to this epitope develop may be useful in tailoring alternate therapeutic interventions.

IgG1 KZ52 (Ebola glycoprotein-specific) antibody (28) was provided by Dr. Dennis R. Burton (The Scripps Research Institute, La Jolla, Calif., USA). To confirm the sequence of the peptide peaks identified by epitope excision mass spectrometry, tandem mass spectrometry (MS/MS) was performed. One μg of soluble gp120 MN (ImmunoDiagnostics, Inc., Woburn, Mass.) was digested with either trypsin or glu-C and subjected to matrix-assisted laser desorption/ionization quadrupole time of flight mass spectrometry (MALDI QqTOF).

Antigen capture ELISA was carried out by first coating 96 well plates (NUNC, Mississauga, ON) with 2.5 μg/ml IgG1 b12 at 4° C. overnight. Plates were washed with phosphate buffered saline (PBS) 0.05% Tween 20 and blocked with PBS containing 0.17% bovine serum albumin. Plates were washed and incubated at 4° C. overnight with serially diluted biotinylated peptides (Nterm and scrambled peptides) corresponding to the amino terminal region of gp120 N-term (biotin-LWVTVYYGVPVWKEATTTLFCASDAK), and a scrambled peptide control (biotin-VWCAPLVYWTSTGELAVDKFVTATYK) (United Biochemical Research, Inc., Seattle, Wash.). Plates were washed and incubated at 37° C. for 45 minutes with streptavidin alkaline phosphatase (Jackson ImmunoResearch Laboratories, Inc. West Grove, Pa.) then washed again and incubated with MgCl₂ diethanolamine substrate buffer plus alkaline phosphatase yellow (pNPP) (Sigma, Oakville, ON.) at room temperature. Plates were read on a Spectramax Plus (Molecular Devices, Sunnyvale, Calif.) at 405 nm. Competition ELISAs were performed similar to the antigen capture test, but serially diluted soluble gp120 IIIB (ImmunoDiagnostics, Inc., Woburn, Mass.) was added to the plate for 1 hour at 37° C., non-bound gp120 was washed away, and then 20 μg/ml peptide was added as above.

Western blot analysis was carried out as follows. One μg of gp120 or BSA was run on a 7.5% SDS-PAGE minigel. The gel was blotted onto a nitrocellulose membrane by Transblot semi-dry transfer cell (Bio-Rad, Mississauga, ON.), blocked, and IgG1 b12 was added at 0.25 μg/ml and incubated for 2 hours at 37° C. The blot was washed with PBS Tween 20 and HRP-sheep anti-human antibody (The Binding Site, San Diego, Calif.) was incubated on the blots. Blots were washed and detected by ECL Advance western blot detection system (Amersham Bioscience, Baie d'Urfe, PQ.).

For immunogenicity studies, 4 groups of 4 BALB/c mice (16 total) were immunized intraperitoneally (i.p.) 5 times over 2 months. The first group of mice received PBS plus Freund's adjuvant in each immunization, and the second group received 10-50 kg gp120. The third and fourth groups received 10 μg biotinylated peptide (group 3 received N-term, group 4, scrambled) linked to avidin. Biotinylated peptides were linked to avidin (Zymed Laboratories, Inc., San Francisco, Calif.) by incubating them at a 1:1 molecular ratio for 30 minutes at 37° C. before inoculation.

ELISAs to detect Ab responses in mice were carried out by coating 96 well plates with 2.5 μg/ml gp120 or 5 μg/ml biotinylated peptide overnight at 4° C. Blood was obtained by venous tail puncture, serum was obtained by collecting the supernatant of blood that had been incubated at 4° C. for one hour and centrifuged in an Eppendorf microcentrifuge (Centrifuge 5417C, Brinkmann Instruments, Ltd. Mississauga, ON) twice for 30 minutes at maximum speed. Serum was diluted at 1/50 down in doubling dilutions and incubated on plates for 2 hours at 37° C., washed, and HRP-goat anti-mouse secondary antibody (Southern Biotechnology Associates, Inc., Birmingham, Ala.) was added. ABTS substrate (Roche Diagnostics Canada, Laval, PQ) was used for detection. Plates were read at 405 nm on the Spectramax Plus.

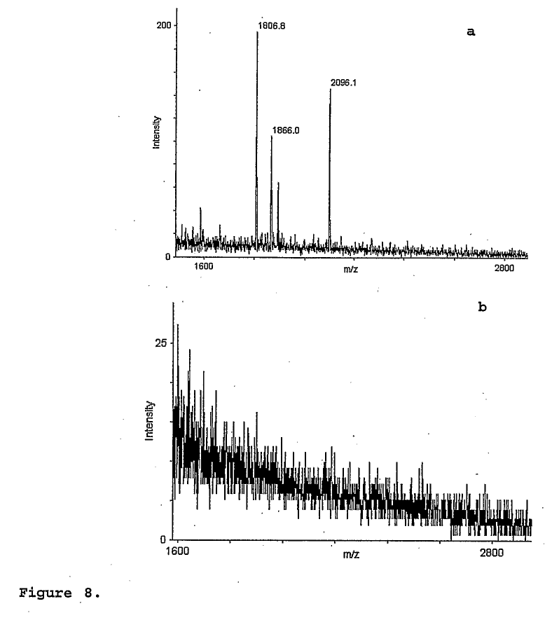

In order to confirm that the mass spectrometry epitope mapping experiments specifically revealed peptides involved in interactions with the variable region of IgG1 b12, control experiments were performed. A non-HIV specific anti-Ebola isotype matched antibody KZ52 was used for such experiments. The KZ52 antibody shares the same constant region and framework region as IgG1 b12, but differs at the variable region (Fv) responsible for binding antigen. This first set of experiments was performed using glu-C endoprotease to digest whole gp120 bound to antibodies. FIG. 8 (a) shows epitope excision mapping results of IgG1 b12 and KZ52 revealing the N-terminal specificity of IgG1 b12 (m/z value 1806.8, 1866.0 and 2096.1); whereas KZ52 (b) lacks these peaks. The masses of the 3 peaks correspond to the earlier identified linear sequence TEKLWVTVYYGVPVWKEATTTLFCASDAK located near the amino terminus of gp120.

FIG. 9 confirms the results observed in FIG. 8. Epitope excision mapping using the endoprotease trypsin shows specific peaks for IgG1 b12 (a) at 1357 and 1609 (roughly), whereas the KZ52 antibody (b) revealed no specific gp120 peptide epitopes. These peaks correspond to the earlier identified linear sequence KLWVTVYYGVPVWKEATTTLFCASDAK, again demonstrating the recognition of IgG1 b12 to the amino terminus of gp120.

MALDI QqTOF mass spectrometry epitope mapping identifies peptide masses that can then be assigned an amino acid sequence based on a theoretical digest of gp120. In order to confirm the predicted sequences observed upon trypsin and glu-C digestion, digested soluble gp120 was subjected to MS/MS sequencing. Soluble gp120, and not antibody-bound gp120 fragments, were used for confirmation because MALDI QqTOF epitope excision mapping yields peptide quantities high enough to be detected, but too low to sequence. Through tandem mass spectrometry, the gp120 peak masses 1357, 1609 (trypsin digest) and 2097 (glu-C digest) sequences were confirmed. This confirmation came by matching more than 50% of the theoretical peaks generated by MS/MS to the actual ones. Although many amino acids in the sequences of the peaks 1807 and 1867 were able to be verified, their numbers of matching peaks were less than 50%. These results support the predicted sequence of LWVTVYYGVPVWKEATTTLFCASDAK for the gp120 amino-terminal epitope of IgG1 b12.

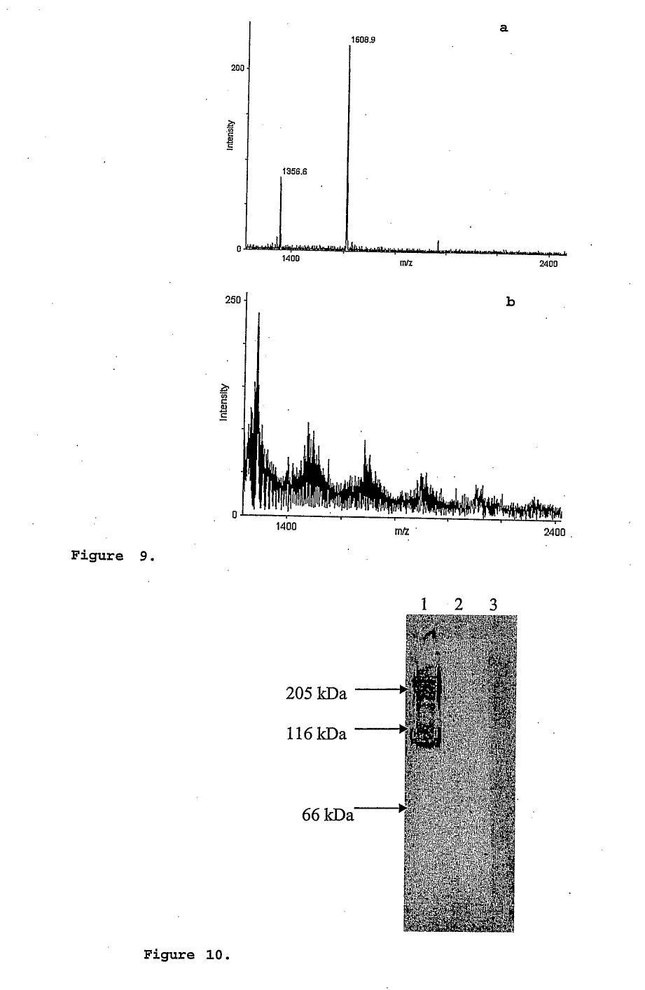

To confirm the linear nature of the IgG1 b12 epitope, we conducted a Western blot analysis of the ability of IgG1 b12 to bind denatured gp120. Analysis of the blot shows IgG1 b12-gp120 recognition bands at approximately 116 kDa (monomeric gp120) and 230 kDa (corresponding to dimeric gp120) in FIG. 10, lane 1. No detectable bands were observed in the second lane, consisting of secondary antibody (sheep anti-human IgG) only. No detectable bands were observed In the third lane, consisting of IgG1 b12 reaction to an irrelevant antigen (BSA-66 kDa).

To confirm that IgG1 b12 recognizes the amino terminal sequence of gp120, ELISA experiments were carried out. A biotinylated peptide matching the identified sequence was tested for binding by antigen capture ELISA. As a control, a biotinylated peptide consisting of the same amino acid sequence as the amino terminal sequence but in a random order (scrambled peptide) was also tested for binding. Results show that the gp120 peptide bound IgG1 b12, and this binding was dose dependant. The binding of the scrambled peptide was significantly lower, and did show dose-dependant binding as did the N-term peptide. This experiment strengthens the evidence that IgG1 b12 recognizes N-term sequence on gp120.

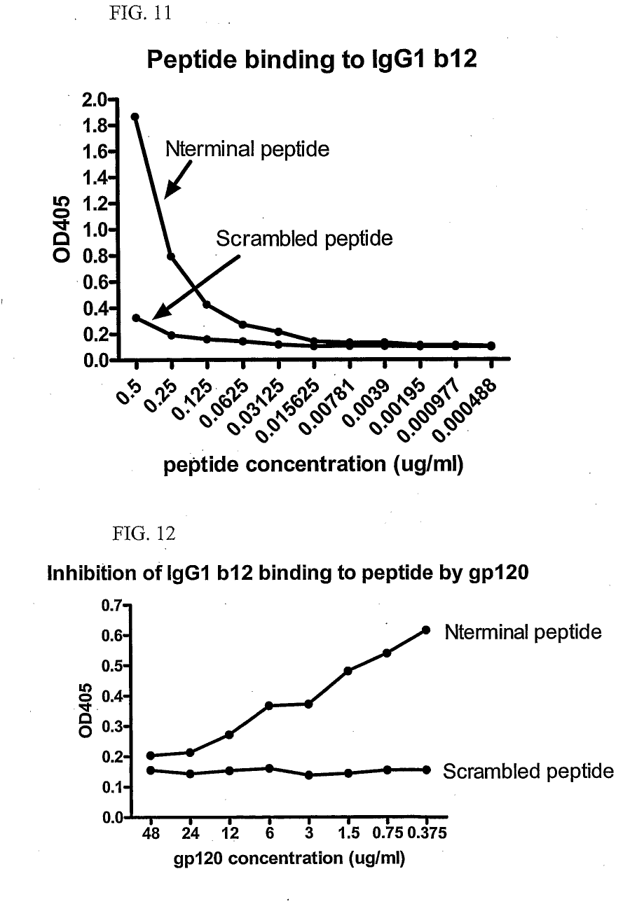

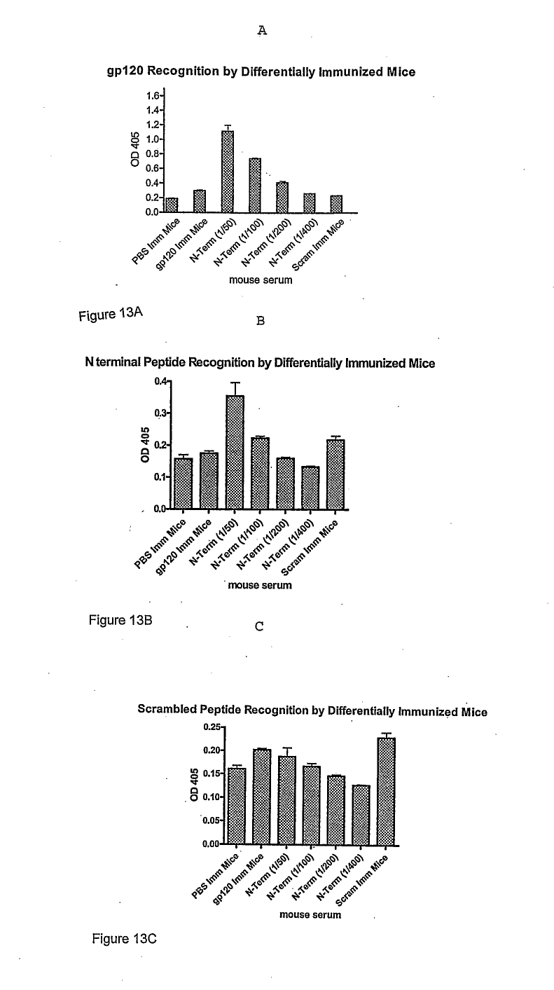

To substantiate the evidence that IgG1 b12 targets the amino terminus of gp120, we assessed the ability of whole gp120 to compete with peptide for binding to IgG1 b12 by ELISA. We incubated plate-bound IgG1 b12 with soluble gp120 before the addition of biotinylated peptide. As evident in FIG. 11, the addition of soluble gp120 blocks peptide binding, and that this binding is gp120 dose dependant.

To determine the immunogenicity of the Nterm peptide, we performed mouse immunizations. Four groups of mouse were given i.p. injections of adjuvant alone, gp120, amino-terminal peptide, or scrambled peptide. Immunizations were administered at days 1, 15, 30, 45 and 60. Testing of the sera collected at day 67 shows that the serum of mice immunized with the N-terminal peptide contains antibodies capable of binding both whole gp120 and N-terminal peptide (FIG. 13, A and B respectively) compared to control mice. The N terminal peptide was therefore immunogenic in the mouse model.

The mass spectrometry control experiments (FIGS. 8 and 9) validate the mass spectrometry results that IgG1 b12 specifically recognizes gp120 at a sequence located near the amino terminus of the whole gp120 molecule. Since the control KZ52 antibody and IgG1 b12 share the same constant region (Fc), but differ in their variable regions (Fv), the differences in their recognition of gp120 by mass spectrometry are attributable only to specific interactions occurring at the antibody Fv. In this set of experiments, the control antibody used, IgG1 KZ52 recognizes specifically Ebola glycoprotein, and not the HIV glycoprotein gp120. The negative IgG1 KZ52 mass spectrometry results indicate that the nature of the interaction between IgG1 b12 and gp120 are highly specific and localized at the antibody variable region. These results solidify and confirm the original mass spectrometry results.

MALDI QqTOF epitope mapping yields peptide peaks that large enough to detect, but too small to sequence. We therefore confirmed the sequence through MS/MS of soluble gp120, a commonly utilized practice. By MS/MS, both trypsin peaks corresponding to the sequences LWVTVYGVPVWK (1357) and EATTTLFCASDAK (1609) were confirmed, and the glu-C digested peak TEKLWVTVYYGVPVWKE (2097) was also confirmed. Taken together these data confirm that the MALDI QqTOF identified peaks are present in soluble, digested gp120 and correspond to the epitopes recognized by IgG1 b12.

Previously, it has been suggested that the gp120 epitope for IgG1 b12 is conformational (7). Contrary to the accepted idea that IgG1 b12 binds a conformation-dependant epitope, the mass spectrometry mapping uncovered an amino acid sequence that was, in fact, linear and located at the amino terminus of gp120. It has yet to be shown whether IgG1 b12 binds denatured gp120. The binding of IgG1 b12 to denatured gp120 suggests that the previously described IgG1 b12-CD4 binding site interactions are incorrect. While the mass spectrometry results do not prove that gp120 conformation is irrelevant to IgG1 b12 binding, it is suggested. The Western blot experiment was performed to back up the evidence that IgG1 b12 binds a linear portion of gp120. That these results were positive corroborates with our mass spectrometry findings. While this does not disprove a conformational component to the interaction of b12 and gp120, what it suggests in conjunction with the mass spectrometry is that there is enough strength in the interactions between b12 and the linear portion at the amino terminus of gp120 to maintain binding over many stringent washes and even endoprotease digestion. We argue that the Nterm sequence on gp120 is the true epitope recognized by IgG1 b12.

To provide further data that supports the IgG1 b12 interaction with the amino terminus of gp120 we tested that ability of IgG1 b12 to bind a biotinylated N-term synthetic peptide via ELISA. The antigenicity of the N terminal peptide was tested. As the synthetic peptide was biotinylated, we used an antigen capture ELISA to detect antibody-peptide interactions. ELISA plates were coated with IgG1 b12 and biotinylated peptide was added and detected for directly. The Nterm peptide bound IgG1 b12 4 times better than the scrambled peptide, in a dose-dependant manner. The scrambled peptide results did not dilute out as did the N terminal peptide signal, indicating non-specific background interactions. We then performed an ELISA where peptide binding to IgG1 b12 was first blocked by the addition of gp120. Addition of gp120 inhibited N-term peptide IgG1 b12 interactions in a dose dependednt manner suggesting that gp120 specifically interferes with N-term peptide binding. This confirms that the area of the variable region on IgG1 b12 that binds gp120 overlaps the area that binds the Nterm peptide. Antigen capture and blocking tests further strengthen the suggestion that IgG1 b12 binds specifically to the amino terminal sequence LWVTVYYGVPVWKEATTTLFCASDAK.

The goal of the mouse immunizations was to test the immunogenicity of the peptide in an animal model—that is to determine if the peptide can elicit antibodies in an in vivo situation. Mice are commonly and easily used as a first step for testing the capability of an antigen to elicit responses in a live animal model. Our mouse data shows that amino terminal peptide was able to elicit antibodies that were able to bind both N terminal peptide and whole gp120. Background-level responses only were seen on the scrambled peptide-coated plates. The N terminal peptide is immunogenic. The positive mouse data also suggests that the Nterm peptide can be exploited as a vaccinogen.

Given that the amino terminus is the binding site of a powerfully neutralizing anti-HIV antibody, it follows that immunization with that amino-terminal sequence will elicit antibody production, and that these antibodies will be HIV-neutralizing. IgG1 b12-like antibodies should block HIV infection of cells in neutralization assays. A future direction will be to perform neutralization assays with the mouse serum generated as above. Briefly, cells will be incubated with dilutions of HIV-1 and mouse serum and infection will be measured by p24 and 13 gal production. We expect that like the monoclonal antibody IgG1 b12, the mouse serum will block HIV-1 infection in vitro. The next step will be to adapt the N-term peptide for human testing and eventually human vaccine phase trials.

The serum generated above will be tested for potency of neutralization of live HIV-1 in vitro cell culture assays. Basically, serum generated above will be incubated with laboratory strains and primary isolates of HIV-1 before the virus is used to infect a variety of susceptible cell lines. The ability to block HIV-1 infection will be determined via HIV-1 p24 protein production and compared to non-specific control antibody preparations. We can also test for the ability of antibodies specific for the described epitope to inhibit transcytosis (the ability of HIV-1 viruses to pass across stratified cell layers) as previously described (27). Basically the ability of antibodies to block this process will be compared to non-specific controls antibodies.

If antibody responses against the described epitope appear to be capable of generating protective responses in vitro (tested above), the ability of the peptide to protect against HIV infection will be assessed in one of two in vivo animal models currently used in HIV-1 challenge studies (14, 15). Basically animals will be vaccinated with the appropriate adjuvant a number of times. They will then be challenged with live HIV-1 and their susceptibility to infection will be assessed at the appropriate time for the animal model involved.

For the HIV-1 neutralization assays, briefly, TZM-b1 cells, a cell line derivative expressing CD4, CXCR4, and CCR5, and firefly luciferase upon infection with HIV were seeded at a density of 3×10³ cells/96-well plate (J. Biol. Chem. 2005; 280: 4095-4101). The next day cells were treated with mouse serum at different concentrations, and one thousand infectious units/well (as determined on TZM-b1 cells) of HIV-1 strains IIIB, SF162, and QH0692 were used to challenge the cells, and 2 days later the cells were lysed and the activity of firefly luciferase activity was determined (Steady-Glo luciferase system, Promega). Because of the induction of firefly luciferase upon infection, the reduction of the relative light units detectable correlates with the inhibition of infection by mouse serum. The viability of the cells was not affected by the addition of serum. The serum dilutions are listed in table 2.

Neutralization results show that the serum of mice immunized with the Nterm peptide can block HIV-1 infection of cells in vitro. The Nterm-specific serum neutralized HIV-1 strain IIIB 18.6 times better that the scrambled peptide-specific serum and 8 times better than mock immunization serum. These results indicate that the N-terminal peptide can elicit HIV-1-specific antibodies that can block infection in an in vitro model.

Minimal to moderate neutralization was noted against the other two HIV strain tested. The slightly high background results seen in the PBS-immunized groups are not surprising, as mouse serum historically displays high background levels in this assay (D. Montefiori, personal communication). These results are a good predictor of the serum antibodies' neutralizing capabilities in vivo.

Binding of synthetic peptides to b12 will assessed by ELISA and other EIA based methods as described and will be done to confirm that this epitope binds to the b12 Mab under approachable physiologic conditions in vivo. This information will be used to design a panel of synthetic peptides that can be used to assess the minimal inhibitory peptide epitope, or peptide sequence that will inhibit binding of b12 to the described epitope. These minimal epitopes will be assessed for their ability to interfere with b12/gp120 binding using ELISA based methods.

The ability of the minimal inhibitory peptide epitope (identified above) to block HIV-1 infection will be determined essentially as previously described, but using peptides corresponding to the identified epitope rather than serum, or MAb's to inhibit HIV-1 infection of the target cells.

The ability of the minimal inhibitory peptide epitope (identified above) to block HIV-1 transcytosis will be determined essentially as previously described, but using peptides corresponding to the identified epitope rather than serum, or MAb's to inhibit HIV-1 transcytosis across target cells.

One further application of the invention is detecting the presence of Ab capable of recognizing described epitope as a means of correlating or analyzing HIV disease progression and/or resistance to infection by HIV-1. This would be the basis for determining if knowledge of reactivity to the described epitope (diagnostic usage) could be useful in altered therapeutic intervention. For example, in a well-described cohort of sex workers from Nairobi, Kenya we have identified groups of HIV-1 infected individuals who progress to AIDS at different rates. The presence of humoral antibody responses to the described epitope may act as a marker for disease progression (i.e. individuals who have this, reactivity may be more likely to progress at a slower rate to AIDS and death). This may be a useful diagnostic tool to aid in the treatment and prescription using HIV-1 anti-retroviral drugs. We will assess reactivity to this epitope in 3 groups of HIV infected individuals, HIV rapid progressors (those who develop AIDS within 3 years), normal progressors (AIDS within 5-7 years), and long-term non-progressors (no AIDS for >10 years).

While the preferred embodiments of the invention have been described above, it will be recognized and understood that various modifications may be made therein, and the appended claims are intended to cover all such modifications which may fall within the spirit and scope of the invention.

### REFERENCES

1. Papac, D. I., Hoyes, J., and K. B. Tomer. 1994. Epitope mapping of the gastrin-releasing peptide/anti-bombesin monoclonal antibody complex by proteolysis followed by matrix-assisted laser desorption ionization mass spectrometry. Protein Sci. 3(9):1485-92.
2. Parker, C. E., Papac, D. I., Trojak, S. K., and K. B. Tomer. 1996. Epitope mapping by mass spectrometry Determination of an epitope on HIV-1IIIB p26 recognized by a monoclonal antibody. J. Immunol. 157:198-206.
3. Parker, C. E., and K. B. Tomer. 2000. Epitope mapping by a combination of epitope excision and MALDI-MS. Methods in Molecular Biology, Vol. 146: Protein and Peptide Analysis. New Mass Spectrometric Applications. J. R. Chapman, Ed. Humana Press, Totowa, N.J. 185-199.
4. Hochleitner, E. O., Gorny, M. K., Zolla-Pazner, S., and K. B. Tomer. 2000. Mass spectrometric characterization of a discontinuous epitope of the HIV protein HIV-gp120 recognized by the human monoclonal antibody 1331A. J. Immunol. 164:4156-61.
5. Collet, T. A., Roben, P., O'Kennedy, R., Barbas III, C. F., Burton D. R., and R. A. Lerner. 1991. A large array of human monoclonal antibodies to type I human immunodeficiency virus from combinatorial libraries of asymptomatic seropositive individuals. Proc. Natl. Acad. Sci. USA. 88:10134-7.
6. Barbas III, C. F., Bjorling, E., Chiodi, F., Dunlop, N., Cababa, D., Jones, T. M., Zebedee, S. L., Persson, M. A. A., Nara, P. L., Norrby, E., and D. R. Burton. 1992. Reombinant human Fab fragments neutralize human type 1 immunodeficiency virus in vitro. Proc. Natl. Acad. Sci. USA. 89:9339-43.
7. Saphire, E. O., Parren, P. W., Pantophlet, R., Zwick, M. B., Morris, G. M., Rudd, P. M., Dwek, R. A., Stanfield, R. L., Burton, D. R., and I. A. Wilson. 2001 Crystal structure of a neutralizing human IGG against HIV-1: a template for vaccine design. Science. 10;293(5532):1155-9.
8. Kwong, P. D., Wyatt, R., Robinson, J., Sweet, R. W., Sodroski, J., and W. A. Hendrickson. 1998 Structure of an HIV gp120 envelope glycoprotein in complex with the CD4 receptor and a neutralizing human antibody. Nature. 18;393(6686):648-59.
9. Frankel, S. S., Steinman, R. M., Michael, N. L., Ratto Kim, S., Bhardwaj, N., Pope, M., Louder, M. K., Ehrenberg, P. K., Parren, P. W. H. I., Burton, D. R., Katinger, H., VANCott, Robb, M. L., Birx, D. L., and J. R. Mascola. 1998. Neutralizing monoclonal antibodies block human immunodeficiency virus type I infection of dendritic cells and transmission to T cells. J. Virol. 72(12):9788-94.
10. Ugolini, S., Mondor, I., Parren, P. W. H. I., Burton, D. R., Katinger, H., Tilley, S. A., Klasse, P. J., and Q. J. Sattenau. 1997. Inhibition of virus attachment to CD4⁺ target cells is a major mechanism of T cell line-adapted HIV-1 neutralization. J. Exp. Med. 186(8):1287-98.
11. Gaudin, M. C., Allaway, G. P. Maddon, P. L., Barbas III, C. F., Burton, D. R., and R. A. Koup. 1996. Effective ex vivo neutralization of human immunodeficiency virus type 1 in plasma by recombinant immunoglobulin molecules. J. Virol. 70(4):2586-92.
12. Gaudin, M. C., Weir, R., Barbas III, C. F., Burton, D. R., and R. A. Koup. 1997. Passive immunization with a human monoclonal antibody protects hu-PBL-SCID mice against challenge by primary isolates of HIV-1.
13. Lewis, A. D., Chen, R., Montefiori, D. C., Johnson, P. R., and K. R. Clark. 2002. Generation of neutralizing activity against human immunodeficiency virus type 1 in serum by antibody gene transfer. J. Virol. 76(17):8769-75.
14. Parren, P. W. H. I., Ditzel, H. J., Gulizia, R. J., Binley, J. M., Barbas III, C. F., Burton, D. R., and D. E. Mosier. 1995. Protection against infection in hu-PBL-SCID mice by passive immunization with a neutralizing human monoclonal antibody against the gp120 CD4-binding site. AIDS 9:F1-F6.
15. Parren, P. W. H. I., Marx, P. A., Hessell, A. J., Luckay, A., Harouse, J., Cheng-Mayer, C., Moore, J. P., and D. R. Burton. 2001. Antibody protects macaques against vaginal challenge with a pathogenic R5 simian/human immunodeficiency virus at serum levels giving complete neutralization in vitro. J. Virol. 75(17):8340-7.
16. Crawford, J. M., Earl, P. L., Moss, B., Reimamm, K. A., Wyand, M. S., Manson, K. H., Bilska, M., Zhou, Z. T., Pauza, C. D., Parren, P. W. H. I., Burton, D. R., Sodrowski, J. G., Letvin, N. L., and D. C. Montefiori. 1999. Characterization of primary isolate-like variants of simian-human immunodeficiency virus. J. Virol. 73(12):10199-207.
17. Kessler II, J. A., McKenna, P. M., Emini, E. A., Chan, C. P., Patel, M. D., Gupta, S. K., Mark III, G. E., Barbas III, C. F., Burton, D. R., and A. J. Conley. 1997. Recombinant human monoclonal antibody IgG1b12 neutralizes diverse human immunodeficiency virus type 1 primary isolates. AIDS Res. Hum. Retro. 13(7):575-82.
18. Moore, J. P., Cao, Y., Quing, L., Sattentau, Q. J., Pyati, J., Koduri, R., Robinson, J., Barbas III, C. F., Burton, D. R., and D. H. Ho. 1995. Primary isolates of human immunodeficiency virus type 1 are relatively resistant to neutralization is not predicted by studies with monomeric gp120. J. Virol. 69(1):101-9.
19. Parren, P. W. H. I., Fisicaro, P., Labrijn, A. F., Binley, J. M., Yang, W-P. Ditzel, H. J., Barbas III, C. F., and D. R. Burton. 1996. In vitro antigen challenge of human antibody libraries for vaccine evaluation: the human immunodeficiency virus type 1 envelope. J. Virol. 73(13):9046-50.
20. Trkola, A., Pomales, A. B., Yuan, H., Korber, B., Maddon, P. J., Allaway, G. P., Katinger, H., Barbas III, C. F., Burton, D. R., Ho, D. D., and J. P. Moore. 1995. Cross-c;ade neutralization of primary isolates of human immundeficiency virus type 1 by human monoclonal antibodies and tetrameric CD4-IgG. J. Virol. 69(11);6609-17.
21. Verrier, F., Nádas, A., Gorny, M. K., and S. Zolla-Pazner. 2001. Additive effects characterize the interaction of antibodies involved in neutralization of the dualtropic human immunodeficiency virus type 1 isolate 89.6. J. Virol. 75(19):9177-86.
22. Xiang, S-H., Kwong. P. D., Gupta, R., Rizzuto, C. D., Casper, D. J., Wyatt, R., Wang, L., Hendrickson, W. A., Doyle, M. L., and J. Sodrowski. 2002 Mutagenic stabilization and/or disruption of a CD4-bound state reveals distinct conformations of the human immunodeficiency virus type 1 gp120 envelope glycoprotein. J. Virol. 76(19):9888-99.
23. Xu W., Smith-Franklin, B. A., Li, P. L., Wood, C., He, J., Du, Q., Bhat, G. J., Kankasa C., Katinger, H., Cavacini, L. A., Posner, M. R., Burton D. R., Chou, T. C., and R. M. Ruprect. 2001. Potent neutralization of primary human immunodeficiency virus clade C isolates with a synergistic combination of human monoclonal antibodies raised against clade B. J. Hum. Virol. 4(2):55-61.
24. Zwick, M. B., Wang, M., Poignard, P., Stiegler, G., Katinger, H., Burton, D. R., and P. H. H. I. Parren. 2001. Neutralization synergy of human immunodeficiency virus type 1 primary isolates by cocktails of broadly neutralizing antibodies. J. Virol. 75(24):12198-208.
25. Burton, D. R. 2002. Antibodies, viruses and vaccines. Nat. Rev. Immunol. 2:706-13.
26. Fowke, K. R., Nagelkerke, N. J., Kimani, J., Simonsen, J. N., Anzala, A. O., Bwayo, J. J., MacDonald, K. S., Ngugi, E. N., and F. A. Plummer. 1996. Resistance to HIV-1 infection among persistently seronegative prostitutes in Nairobi, Kenya. Lancet 16;348(9038):1347-51.
27. Devito, C., Hinkula, J., Kaul, R., Lopalco, L., Bwayo, J. J., Plummer, F., Clerici, M., and K. Broliden. 2000. Mucosal and plasma IgA from HIV-exposed seronegative individuals neutralize a primary HIV-1 isolate. AIDS 14(13):1917-20
28. Toshiaki Maruyama, Luis L. Rodriguez, Peter B. Jahrling, Anthony Sanchez, Ali S. Khan, Stuart T. Nichol, C. J. Peters, Paul W. H. I. Parren, and Dennis R. Burton. J. Virol. 1999. Ebola virus can be effectively neutralized by antibody produced in natural human infection. 73: 6024-6030.

TABLE 1

Tandem mass spectrometry sequencing confirms 3 of the 5 sequences
derived from epitope mapping. Peaks 1357, 1609, 1807, 1867 and
2097 were selected from digested gp120 complete spectra for MS/MS
on the MALDI QqTOF. The a, b. and y ionic fragmentation peptide
masses were measured and percent match was calculated. Tabulated
are the y ion fragment matches, which are representative of the
three forms of fragmentation.

| | | | % | sequence |
|---|---|---|---|---|
| Trypsin | sequence | y ion matched sequences | match | match |
| 1357 | EATTTLFCASDAK | E, EAT, EATT, EATTTL, | 54 | yes |
| | | EATTTLF, EATTTLFC, | | |
| | | EATTTLFCA, EATTTLFCAS, | | |
| | | EATTTLFCASDA, EATTTLFCASDAK | | |
| 1609 | LWVTVYYGVPVWK | L, LWV, LWVT, LWVTV, | 67 | yes |
| | | LWVTVYLWVTVY, LWVTVYY, | | |
| | | LWVTVYYG, LWVTVYYGV, | | |
| | | LWVTVYYGVP, LWVTVYYGVPV, | | |
| | | LWVTVYYGVPVW, LWVTVYYGVPVWK | | |
| | | | % | sequence |
| glu-c | sequence | y ion matched sequences | match | match |
| 1807 | ATTTLFCASDAKAYDTE | A, ATTTL, ATTTLFCA, | 25 | no |
| | | ATTTLFCASDAK, ATTTLFCASDAKAYD | | |
| 1867 | KLWVTVYYGVPVWKE | K, KL, KLWVTVYYG, KLWVTVYYGVP, | 38 | no |
| | | KLWVTVYYGVPVW | | |
| 2097 | TEKLWVTVYYGVPVWKE | T, TEK, TEKLWV,TEKLWVT, | 66 | yes |
| | | TEKLVTV, TEKLWVTVY, TEKLWVTVYY, | | |
| | | TEKLWVTVYYG, TEKLWVTVYYGV, | | |
| | | TEKLWVTVYYGVP, TEKLWVTVYYGVPV, | | |
| | | TEKLWVTVYYGVPVW, TEKLWVTVYYGVPVWK | | |

TABLE 2
Neutralization assay results for mice immunized with the N terminal
epitope. Serum was isolated and from 4 groups of 4 mice
immunized with PBS (P), gp120(G), N terminal peptide (N),
or Scrambled peptide (S), then pooled by group. Neutralizing
ability was tested for both serum collected from a
pre-immunization bleed (P0, G0, N0, S0), and post
immunization (P1, G1, N1, S1). Neutralization ability was
tested against 3 strains of HIV-1 (IIIB, SF162.LS and
QH0692.4) in TZM-b1 cells.

| Sample | IIIB | SF162 | QH0692 |
|---|---|---|---|
| P0 | 34¹ | <20 | 61 |
| P1 | 228 | <20 | 42 |
| G0 | 37 | <20 | 31 |
| G1 | 108 | 26 | 49 |
| N0 | 49 | <20 | <20 |
| N1 | 1,826 | 39 | 88 |
| S0 | 45 | <20 | <20 |
| S1 | 98 | <20 | 21 |

¹Values are the serum dilution at which relative luminescence units (RLUs) were reduced 50% compared to virus control wells (no test sample).

1. A purified polypeptide, the amino acid sequence of which comprises at least 6 contiguous residues of any one of SEQ ID No. 1-6.

2. A method of immunizing an individual against Human Immunodeficiency Virus (HIV) infection comprising administering to an individual a purified polypeptide, the amino acid sequence of which comprises at least 6 contiguous residues of any one of SEQ ID No. 1-6.

3. (canceled)

4. (canceled)

5. A method of preparing an immune globulin effective against Human Immunodeficiency virus comprising:
vaccinating a plurality of donors with a purified polypeptide, the amino acid sequence of which comprises at least 6 contiguous residues of any one of SEQ ID No. 1-6;
isolating plasma from each of said donors after a period of time sufficient to allow production of antibodies against said polypeptide;
pooling the plasma; and
preparing an immune globulin from the pooled plasma.

6. A method of determining a course of treatment for an individual infected with human immunodeficiency virus comprising:
screening a sample from an individual infected with human immunodeficiency virus for antibodies binding to a purified polypeptide, the amino acid sequence of which comprises at least 6 contiguous residues of any one of SEQ ID No. 1-6,
wherein presence of antibodies against said polypeptide indicates that a less aggressive treatment is needed.

7. A method of treating an individual infected or suspected of being infected by human immunodeficiency virus comprising administering to said individual a therapeutically effective amount of a purified polypeptide, the amino acid sequence of which comprises at least 6 contiguous residues of any one of SEQ ID No. 1-6.

8. A method of treating an individual infected or suspected of being infected by human immunodeficiency virus comprising administering to said individual a purified polypeptide, the amino acid sequence of which comprises at least 6 contiguous residues of any one of SEQ ID No. 1-6.


SEQUENCE LISTING (6 sequences)

SEQ ID NO: 1 - protein, 26 aa, Human immunodeficiency virus
LWVTVYYGVPVWKEATTTLFCASDAK

SEQ ID NO: 2 - protein, 12 aa, Human immunodeficiency virus
GVPVWKEATTTL

SEQ ID NO: 3 - protein, 26 aa, Human immunodeficiency virus
  VARIANT (13)..(13): E or K or R
LWVTVYYGVPVWXXAXXXLFCASDAK

SEQ ID NO: 4 - protein, 12 aa, Human immunodeficiency virus
  VARIANT (6)..(6): E or K or R
GVPVWXXAXXXL

SEQ ID NO: 5 - protein, 26 aa, Human immunodeficiency virus
  VARIANT (13)..(13): E or K or R
LWVTVYYGVPVWXXAXXXLFCASDAK

SEQ ID NO: 6 - protein, 12 aa, Human immunodeficiency virus
  VARIANT (6)..(6): E or K or R
GVPVWXXAXXXL